(19) United States
(12) Patent Application Publication
(10) Pub. No.: US 2004/0215313 A1
(43) Pub. Date: Oct. 28, 2004

(54) Stent with sandwich type coating

(76) Inventor: Peiwen Cheng, Santa Rosa, CA (US)

Correspondence Address: FRANK C. NICHOLAS, CARDINAL LAW GROUP, Suite 2000, 1603 Orrington Avenue, Evanston, IL, 60201, US

(21) Appl. No.: 10/827,817

(22) Filed: Apr. 20, 2004

Related U.S. Application Data
(60) Provisional application No. 60/464,612, filed on Apr. 22, 2003.

(51) Int. Cl. A61F 2/06

(52) U.S. Cl. 623/1.11; 623/1.42; 623/1.46; 427/2.25

(57) ABSTRACT

The stent having a sandwich type coating of the present invention provides multiple coating layers, with the first coating layer providing adhesion to the stent frame, the second coating layer providing the main reservoir for drugs or therapeutic agents, and the third coating layer providing a protective coating. The first coating layer or third coating layer can also include drugs or therapeutic agents. The first coating layer can be an organic silane or polymer material. The second and third coating layers can be biodegradable or non-biodegradable polymers, with the second and third coating layers made of the same or different polymers. Different therapies and delivery timing can be achieved by selection of different materials and therapeutic agents for the different coating layers.

### RELATED APPLICATION

This application claims priority to U.S. Provisional Application No. 60/464,612, “Stent with Sandwich Type Coating” to Peiwen Cheng, filed Apr. 22, 2003, the entirety of which is incorporated by reference.

### TECHNICAL FIELD

The technical field of this disclosure is medical implant devices, particularly, a stent having a sandwich type coating.

### BACKGROUND OF THE INVENTION

Stents are generally cylindrical shaped devices that are radially expandable to hold open a segment of a blood vessel or other anatomical lumen after implantation into the body lumen. Stents have been developed with coatings to deliver drugs or other therapeutic agents.

Stents are used in conjunction with balloon catheters in a variety of medical therapeutic applications including intravascular angioplasty. For example, a balloon catheter device is inflated during PTCA (percutaneous transluminal coronary angioplasty) to dilate a stenotic blood vessel. The stenosis may be the result of a lesion such as a plaque or thrombus. After inflation, the pressurized balloon exerts a compressive force on the lesion thereby increasing the inner diameter of the affected vessel. The increased interior vessel diameter facilitates improved blood flow. Soon after the procedure, however, a significant proportion of treated vessels re-narrow, i.e., restenosis occurs. Neointimal hyperplasia, intimal thickening, smooth muscle proliferation, vascular lumen elastic recoil, and vascular remodeling may all contribute to restenosis.

To prevent restenosis, short flexible cylinders, or stents, constructed of metal or various polymers are implanted within the vessel to maintain lumen size. The stents acts as a scaffold to support the lumen in an open position. Various configurations of stents include a cylindrical tube defined by a mesh, interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz and U.S. Pat. No. 5,421,955 to Lau. Balloon-expandable stents are mounted on a collapsed balloon at a diameter smaller than when the stents are deployed. Although the stent injured restenosis rates are 30% lower than balloon (PTCA) injured restenosis rates, 25% of patients have restenosis and need to be revescularzied.

Recently, stents have been used with coatings to deliver drug or other therapy at the site of the stent to treat restenosis. The coating can be applied as a liquid containing the drug or other therapeutic agent dissolved or dispersed in a polymer/solvent matrix. The solvent can be one solvent or a mixture of solvents and the polymer can be copolymers or polymer blend. The liquid coating can be applied by spraying, dipping, brushing, painting, wiping, vapor deposition, plasma deposition, electrostatic deposition, epitaxial growth, combinations thereof, and other methods. The liquid coating then dries to a solid coating upon the stent, the coating forming a drug reservoir in the dried polymer film. Combinations of the various application techniques can also be used.

Problems arise in getting coatings to adhere to stents, particularly stents made of stainless steel. Most coronary stents are made of stainless steel or tantalum and finished by electrochemical polishing for surface smoothness. Surface smoothness is desirable because early research showed that a stent with a rough surface results in more platelet cell adhesion, thrombus, inflammation, and restenosis than a polished stent. The smooth surface poses a challenge to the coating, however. Due to the very different nature of the polymer and the metallic substrate, polymers do not easily adhere to the metallic substrate surface.

Additional problems arise in implanting the stent. The coated stent must go through tortuous coronary vessels to reach the implantation site, so some drug may be prematurely lost through contact with the vessel walls or catheters. In addition to loss, some portion of the coating may be damaged. Loss or damage can result in uncertainty in the delivered drug dosage and require increased drug loading of expensive therapeutic agents to assure an effective dose is delivered. In addition, broken debris from the coating may cause serious embolization.

Because of the difficulties in obtaining coating adherence and maintaining the coating on the stent during implantation, existing stents have been limited to one or two layer coatings. This limits the potential therapies to a single polymer/drug combination for a one layer coating and two polymer/drug combinations for a two layer coating.

U.S. Pat. No. 6,306,176 to Whitbourne et al. issued Oct. 23, 2001, discloses an insertable medical device with an abrasion resistant surface coating. The device surface is an inert surface and is pretreated with plasma or other ionizing pretreatment. The bonding material is polymeric material forming non-covalent bonds with the surface.

U.S. Pat. No. 5,607,475 to Cahalan et al. issued Mar. 4, 1997, and U.S. Pat. No. 5,782,908 to Cahalan et al. issued Jul. 21, 1998, disclose a medical article having a metal or glass surface with the surface having an adherent coating of improved biocompatibility. The coating is made by first applying to the surface an silane compound having a pendant vinyl functionality such that the silane adheres to the surface and then, in a separate step, forming a graft polymer on the surface with applied vinylsilane such that the pendant vinyl functionality of the vinylsilane is incorporated into the graft polymer by covalent bonding with the polymer. Biomolecules may then be covalently attached to the base layer.

European Patent EP0982041 A1 to Tedeschi et al. published on Mar. 1, 2000, discloses coatings in which biopolymers may be covalently linked to a substrate. Coatings disclosed include those that permit coating of a medical device in a single layer, including coatings that permit applying the single layer without a primer. Suitable biopolymers include heparin complexes, and linkage may be provided by a silane having isocyanate functionality.

U.S. Pat. No. 5,735,897 to Buirge issued Apr. 7, 1998, discloses a multi-layer vascular therapeutic-containing prosthesis designed and arranged to “pump” the therapeutics into the blood stream. An inner porous support layer and an outer support layer trap and hold there between a swellable drug or therapeutic-containing layer.

It would be desirable to have a stent having a sandwich type coating that would overcome the above disadvantages.

### SUMMARY OF THE INVENTION

One aspect of the present invention provides a stent having a sandwich type coating to increase coating adherence.

Another aspect of the present invention provides a stent having a sandwich type coating to retain the coating on the stent during implantation.

Another aspect of the present invention provides a stent having a sandwich type coating to allow multiple therapies.

Another aspect of the present invention provides a stent having a sandwich type coating to allow controlled therapy delivery by different sandwich coating designs, such as different therapeutic agents, polymers, coating thicknesses, or coating layers with or without therapeutic agents.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 shows a stent delivery system made in accordance with the present invention.

FIG. 2 shows a stent with sandwich type coating made in accordance with the present invention.

FIG. 3 shows a transverse cross section of a portion of a stent with sandwich type coating made in accordance with the present invention.

FIGS. 4-7 show graphs of exemplary elution rates for stents with sandwich type coating made in accordance with the present invention.

FIG. 8 shows a flow chart of a method of manufacturing a stent with sandwich type coating made in accordance with the present invention.

### DETAILED DESCRIPTION OF THE PRESENTLY PREFERRED EMBODIMENT

The stent having a sandwich type coating of the present invention provides multiple coating layers, with the first coating layer providing adhesion to the stent frame, the second coating layer providing the main reservoir for drugs or therapeutic agents, and the third coating layer providing a protective coating. The first coating layer or third coating layer can also include drugs or therapeutic agents. The first coating layer can be an organic silane or polymer materials. The second and third coating layers can be biodegradable or non-biodegradable polymers, with the second and third coating layers made of the same or different polymers. Different therapies and delivery timing can be achieved by selection of different materials and therapeutic agents for the different coating layers.

In one embodiment, for example, the third layer can incorporate antiplatelet, anti-thrombus, or anti-inflammatory agents that will battle platelet accumulation and thrombus inflammation that can occur in an early stage of restenosis. The second layer can incorporate antirestenosis agents that battle major restenosis. The first layer can incorporate healing promoter agents to promote complete vascular healing.

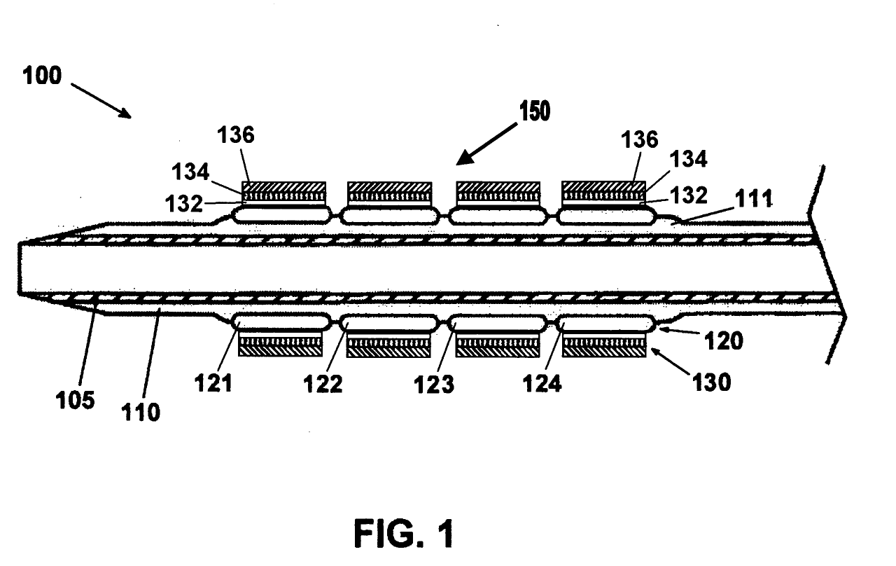

FIG. 1 shows a stent delivery system made in accordance with the present invention. The stent delivery system 100 includes a catheter 105, a balloon 110 operably attached to the catheter 105, and a stent 150 disposed on the balloon 110. The balloon 110, shown in a collapsed state, may be any variety of balloons capable of expanding the stent 150. The balloon 110 may be manufactured from any sufficiently elastic material such as polyethylene, polyethylene terephthalate (PET), nylon, or the like. In one embodiment, the balloon 110 may include retention means 111, such as mechanical or adhesive structures, for retaining the stent 150 until it is deployed. The catheter 105 may be any variety of balloon catheters, such as a PTCA (percutaneous transluminal coronary angioplasty) balloon catheter, capable of supporting a balloon during angioplasty.

The stent 150 comprises a stent frame 120 and a coating 130, the coating 130 comprising a first coating layer 132, a second coating layer 134, and a third coating layer 136. The stent frame 120 may be any variety of implantable prosthetic devices capable of carrying a coating known in the art. In one embodiment, the stent frame 120 may have a plurality of identical cylindrical stent segments placed end to end. Four stent segments 121,122,123, and 124 are shown, and it will be recognized by those skilled in the art that an alternate number of stent segments may be used.

The stent frame 120 is conventional to stents generally and can be made of a wide variety of medical implantable materials, such as stainless steel, tantalum, nitinol, ceramic, nickel, titanium, aluminum and their alloys, polymeric materials, MP35 alloys, MP35N, MP35W, titanium ASTM F63-83 Grade 1, niobium, high carat gold K 19-22, or combinations and alloys of the above. The stent frame 120 can be formed through various methods as well. The stent frame 120 can be welded, laser cut, molded, or consist of filaments or fibers which are wound or braided together in order to form a continuous structure. Depending on the material, the stent can be self-expanding or be expanded by a balloon or some other device. Self-expanding stents can be made of materials such as shape memory metal or temperature memory metal, for example.

The coating 130 can be applied to the stent frame 120 by dipping, brushing, vapor deposition, plasma deposition, electrostatic deposition, epitaxial growth, or spraying the stent frame 120 with a coating liquid, or applying the coating liquid with a combination of methods. The coating layers can be applied as a liquid containing a drug or other therapeutic agent dissolved or dispersed in a polymer/solvent matrix. In another embodiment, the therapeutic agent can be omitted from the coating and the coating included for its mechanical properties.

The coating 130 can be a biodegradable or non-biodegradable polymer. Examples of biodegradable polymers include polycaprolactone, polylactide, polyglycolide, polyorthoesters, polyanhydrides, poly(amides), poly(alkyl 2-cyanocrylates), poly(dihydropyrans), poly(acetals), poly(phosphazenes), poly(dioxinones), trimethylene carbonate, polyhydroxybutyrate, polyhydroxyvalerate their copolymers, blends and copolymers blends and combinations of the above, and the like. Non-biodegradable polymers can be further divided into two classes. The first class is hydrophobic polymers such as polyolefins, acrylate polymers, vinyl polymers, styrene polymers, polyurethanes, polyesters, epoxy, nature polymers, their copolymers, blends and copolymer blends, combinations of the above, and the like. The second class is hydrophilic polymers or hydrogels such as polyacrylic acid, polyvinyl alcohol, poly(N-vinylpyrrolidone), poly(hydroxy, aklymethacrylate), polyethylene oxide, their copolymers, blends and copolymer blends, combinations of the above, and the like.

Suitable solvents that can be used to form the liquid coating include, but are not limited to, water, alcohol, acetone, acetonitrile, ether, methyl ether ketone (MEK), ethyl acetate, tetrahydrofuran (THF), dioxane, chloroform, methylene chloride, xylene, toluene, N,N-dimethylformamide (DMF), dimethyl sulfoxide (DMSO), N,N-dimethylacetamide (DMAC), N-methylpyrrolidone (NMP), combinations of the above, and the like. Suitable therapeutic agents include, but are not limited to antiangiogenesis agents, antiendothelin agents, antimitogenic factors, antioxidants, antiplatelet agents, antiproliferative agents, antisense oligonucleotides, antithrombogenic agents, antibiotics, anti-inflammatory agents, antiinfective agents, antidiabetic agents, antiarteriosclerotics, antiarythmics, calcium channel blockers, clot dissolving enzymes, growth factors, growth factor inhibitors, nitrates, nitric oxide releasing agents, vasodilators, virus-mediated gene transfer agents, agents having a desirable therapeutic application, combinations of the above, and the like. Specific examples of therapeutic agents include abciximab, angiopeptin, colchicine, eptifibatide, heparin, hirudin, lovastatin, methotrexate, Resten-NG (AVI-4126) antisense compound, streptokinase, ticlopidine, tranilast, sulindac, etoposide, podophyllotoxin, 5-fluorouracil, tissue plasminogen activator, trapidil, urokinase, and growth factors VEGF, TGF-beta, IGF, PDGF, and FGF.

The first coating layer 132 can act as a bridge to promote adhesion between the inorganic metal stent frame 120 and the organic second coating layer 134. Typically, the second coating layer 134 can be a carrier for the therapeutic agent, and the third coating layer 136 can be a top coating to protect the underlying layers. The coating 130 is merely exemplary, and it should be recognized that other coating configurations, such as additional coating layers, are possible.

Although the coating 130 is shown schematically on the outside of the stent frame 120, the coating 130 can cover the whole stent frame 120, both inside and outside. In other embodiments, the coating 130 can vary by portion of the stent frame 120, e.g., the individual stent segments 121, 122, 123, and 124 can have different numbers of coating layers, coating layers with different therapeutic agents, or coating layers using different polymers. Those skilled in the art will appreciate that many combinations are possible.

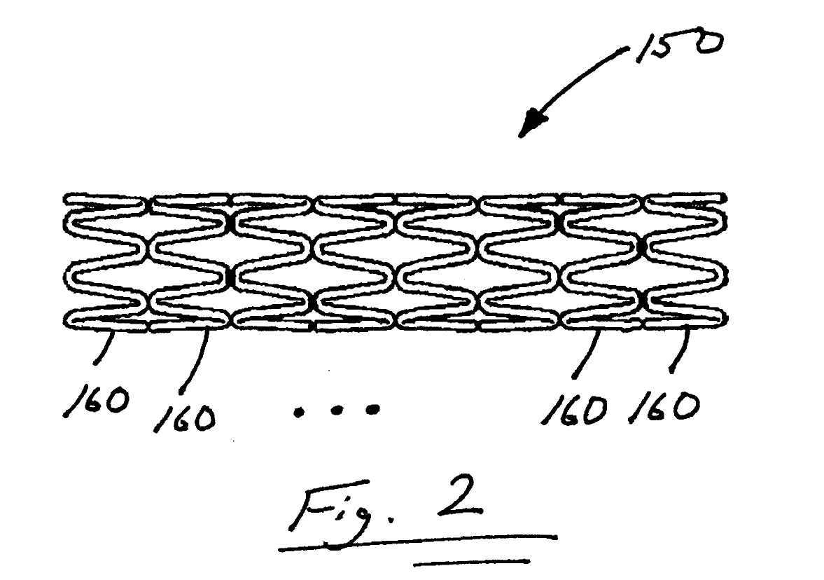

FIG. 2 shows a stent made in accordance with the present invention. The stent 150 comprises a number of segments 160. The pattern of the segments 160 can be W-shaped or can be a more complex shape with the elements of one segment continuing into the adjacent segment. The stent 150 can be installed in the stent delivery system of FIG. 1 for implantation in a body lumen.

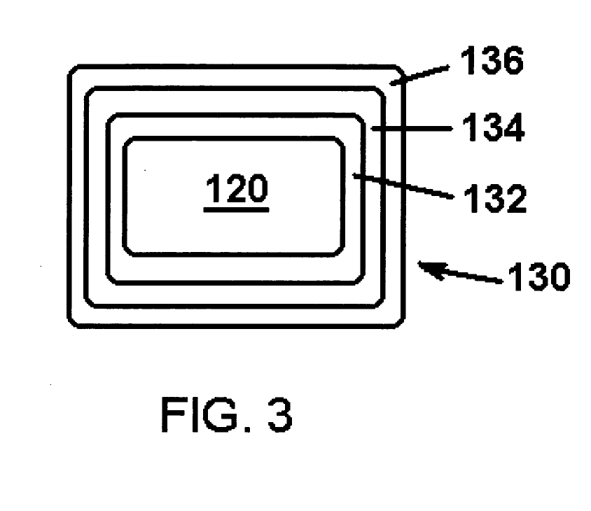

FIG. 3 shows a transverse cross section of a portion of a stent made in accordance with the present invention. A coating 130 is disposed on a stent frame 120, the coating 130 comprising a first coating layer 132, a second coating layer 134, and a third coating layer 136. Typically, the first coating layer 132 can promote adhesion to the stent frame 120, the second coating layer 134 can carry a therapeutic agent, and the third coating layer 136 can protect the underlying coating layers.

The first coating layer 132 can be a primer coating layer, acting as a bridge to promote adhesion between the inorganic metal stent frame 120 and the organic second coating layer 134. The first coating layer 132 can be a thin primer coating of a low molecular weight compounds, such as a silane. The general formula of organic silane is RnSiX₍₄₋ₙ₎. X can be alkoxy, acyloxy, amine, chlorine, or the like, which can react with inorganic substrate of the stent frame 120 to replace a bond between X and Si. R can be an organic radical to bond with polymers of the second coating layer 134 and can be matched with the material used in the second coating layer 134. For example, R can be an acrylate if acrylate polymers are used in the second coating layer, R can be an ester if ester polymers are used in the second coating layer, and R can be an isocynate if polyurethane is used in the second coating layer. Suitable silanes include, but are not limited to, vinyltris(methylethylketoxime)silane, 2-(diphenylphosphino)ethyltriethoxysilane, 3-(1-aminopropoxy)-3,3-dimethyl-1-propenyl-trimethoxysilane, (aminoethylaminomethyl)phenethyltrimethoxysilane, 3-methacryloxypropyltrimethoxysilane, trimethoxysilyl-propyldiethylene-triamine, trichlorovinylsilane, 3-isocyanyopropyltriethoxysilane, 5-hexenyltrimethoxysilane, and the like. Those skilled in the art will appreciate that various silanes can be used depending on the stent frame and second coating layer materials used.

In another embodiment, the first layer coating can be an adhesion promoter coating of a high molecular weight polymer, such as silicone polymers, acrylate polymers, epoxy type polymers, carboxylic polymers, polysulfide, phenolic resin, amino resin, polyurethane, polyvinyl acetate, polyvinyl alcohol, polyvinyl acetal, cyanoacrylate, polyester, polyamide, and combinations thereof. The first coating layer 132 can be applied by spraying, dipping, brushing, painting, wiping, vapor deposition, plasma deposition, electrostatic deposition, epitaxial growth, or combinations thereof. The thickness of the first coating layer 132 can be thick or thin depending on the particular application. In one embodiment, the first coating layer 132 can act as a polymer reservoir containing a drug or therapeutic agent. The therapeutic agent can be gradually eluted through the overlying coating layers or eluted at a later time if the overlying layers biodegrade.

The second coating layer 134 can be a drug reservoir coating layer and typically can be the major reservoir for a drug or therapeutic agent. The relative fraction of polymer to drug can be adjusted depending on the delivery characteristics required: a large quantity of drug with a small quantity of polymer as a binder for rapid delivery, or a small quantity of drug with a large quantity of polymer as a reservoir for a more prolonged delivery. The polymers of the second coating layer 134 can be a biodegradable polymer, or a hydrophobic or hydrophilic non-biodegradable polymer. Examples of biodegradable polymers include polycaprolactone, polylactide, polyglycolide, polyorthoesters, polyanhydrides, poly(amides), poly(alkyl 2-cyanocrylates), poly(dihydropyrans), poly(acetals), poly(phosphazenes), poly(dioxinones), trimethylene carbonate, polyhydroxybutyrate, polyhydroxyvalerate, similar polymers, their blends and copolymers and copolymers blends, and combinations thereof. The non-biodegradable polymers can be further divided into two classes. The first class is hydrophobic polymers suitable for hydrophobic drugs application. The second class is hydrophilic polymers suitable for hydrophilic drug application. Examples of non-biodegradable hydrophobic polymers include polyolefins, polystyrene, polyester, polysulfide, polyurethanes, polyacrylates, silicone polymers, cellulose polymers, polyvinyl polymers, similar polymers, their blends and copolymers, copolymer blends, and combinations thereof. Examples of non-biodegradable hydrophilic polymers include polyvinyl alcohol and its derivatives, polyvinyl pyrrolidone, polyethylene oxide, poly(hydroxy, aklymethacrylate), similar polymers, their blends and copolymers, and combinations thereof. The second coating layer 134 can be applied by spraying, dipping, brushing, painting, wiping, vapor deposition, plasma deposition, electrostatic deposition, epitaxial growth, or combinations thereof.

The third coating layer 136 can be a protective coating layer to act as a top coat, protecting the underlying coating layers from damage or premature loss. The third coating layer 136 can be a polymer alone or can be a polymer loaded with a drug or therapeutic agent. The polymer alone provides a barrier to protect the underlying coating layers. In one embodiment, the third coating layer 136 can include a quantity of drug to compensate for drug loss on the tortuous passage through the coronary vessel prior to implantation. The drug disposed in the third coating layer 136 will be delivered rapidly because the third coating layer 136 is on the outside of the stent next to the vessel wall. In another embodiment, the third coating layer 136 can be used to control the drug elution rate from the underlying coating layers by controlling the diffusivity and thickness of the polymer forming the third coating layer 136. The polymer of the third coating layer 136 can be the same as the polymer of the second coating layer 134, or can be a different polymer. The polymers of the third coating layer 136 can be biodegradable or non-biodegradable. Examples of biodegradable polymers include polycaprolactone, polylactide, polyglycolide, polyorthoesters, polyanhydrides, poly(amides), poly(alkyl 2-cyanocrylates), poly(dihydropyrans), poly(acetals), poly(phosphazenes), poly(dioxinones), trimethylene carbonate, polyhydroxybutyrate, polyhydroxyvalerate, similar polymers, their blends and copolymers, and combinations thereof. The non-biodegradable polymers can be further divided into two classes: hydrophobic polymers and hydrophilic polymers. Examples of non-biodegradable hydrophobic polymers include polyolefins, polystyrene, polyester, polysulfide, polyurethanes, polyacrylates, silicone polymers, cellulose polymers, polyvinyl polymers, similar polymers, their blends and copolymers copolymer blends, and combinations thereof. Examples of non-biodegradable hydrophilic polymers include polyvinyl alcohol and its derivatives, polyvinyl pyrrolidone, polyethylene oxide, poly(hydroxy, aklymethacrylate), similar polymers, their blends and copolymers, and combinations thereof. The third coating layer 136 can be applied by spraying, dipping, brushing, painting, wiping, vapor deposition, plasma deposition, electrostatic deposition, epitaxial growth, or combinations thereof.

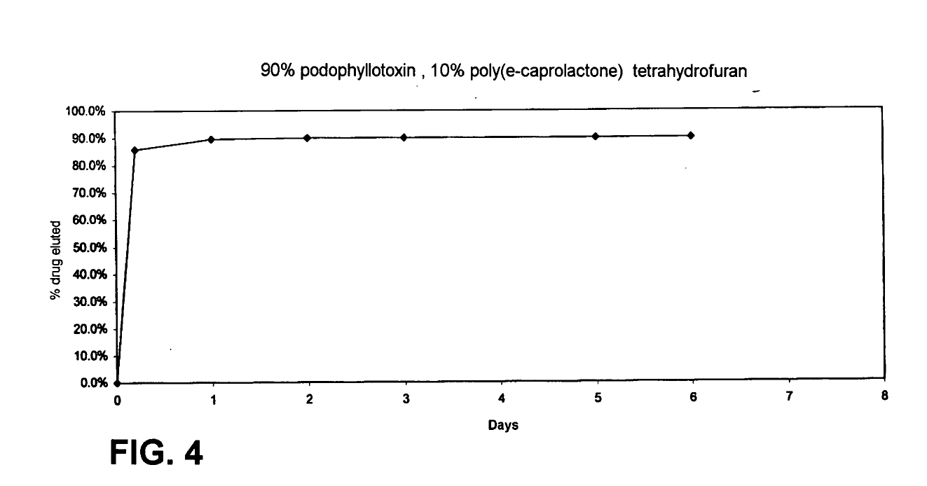

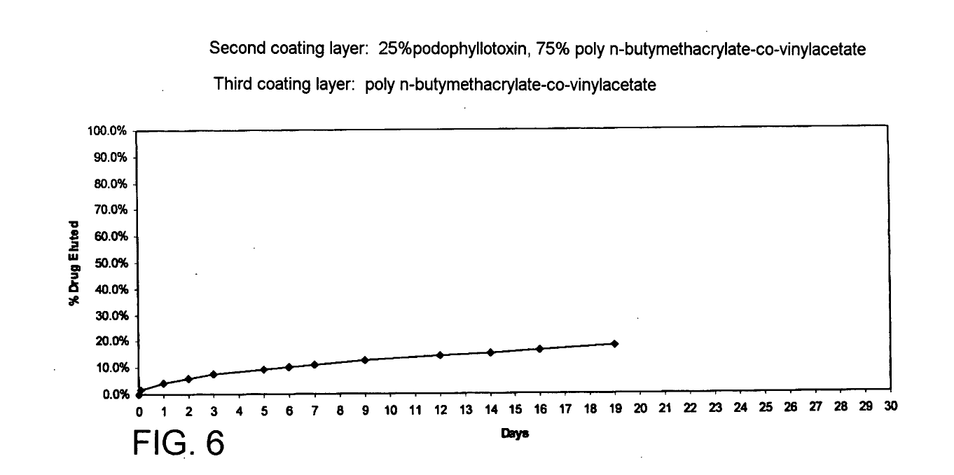

FIGS. 4-7 show the changes in elution rates possible with variation in coating materials and relative position. The examples of FIGS. 4 & 5 show the different elution rates possible for a single coating layer such as can be used for a second or third coating layer. The examples of FIGS. 6 & 7 show the different elution rates possible for different concentrations and different coating layers.

Referring to FIG. 4, 0.2006 g of podophyllotoxin was weighed into a glass bottle. A weight of 0.2010 g of poly(c-caprolactone) was weighed in a weighing pan and transferred into the bottle. A volume of 44.7 ml of tetrahydrofuran (THF) was added to the vial and the bottle shaken until all the drug and polymer dissolved. The solution was applied to stents to form a coating. The experimentally determined elution rate is presented in FIG. 4. The elution rate was relatively fast, reaching 40% elution in less than a day. Is the figure really 90/10 with equal weights podophyllotoxin and poly(ε-caprolactone) from the example explanation?

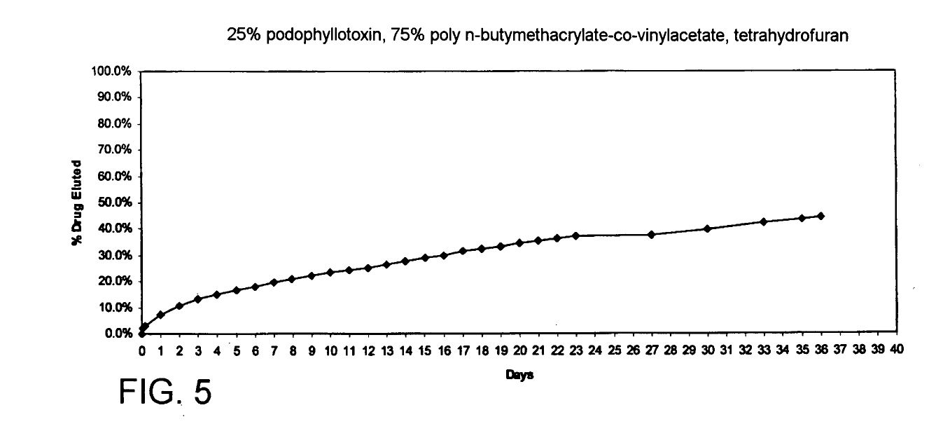

Referring to FIG. 5, 0.2109 g of podophyllotoxin was weighed into a glass bottle. A weight of 0.6345 g of poly n-butymethacrylate-co-vinylacetate, 60:40, was weighed in a weighing pan and transferred into the bottle. A volume of 89 ml of tetrahydrofuran (THF) was added to the bottle and the bottle shaken until all the drug and polymer dissolved. The solution was applied to stents to form a coating. The experimentally determined elution rate is presented in FIG. 5. The elution rate was relatively slow, reaching 40% elution in about 20 days.

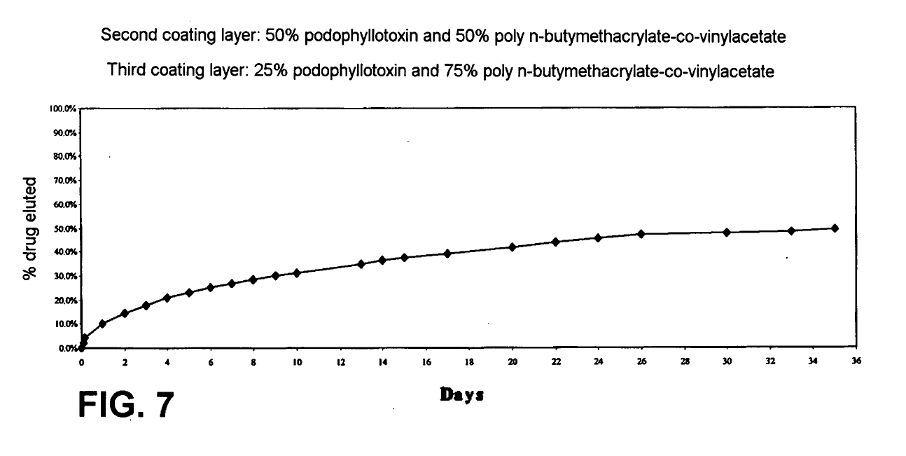

Referring to FIG. 6, a second coating layer with 25% podophyllotoxin and 75% poly n-butymethacrylate-co-vinylacetate was provided with a poly n-butymethacrylate-co-vinylacetate third coating layer. As shown in FIG. 6, the result is a relatively slow and steady elution rate. Referring to FIG. 7, a second coating layer with 50% podophyllotoxin and 50% poly n-butymethacrylate-co-vinylacetate was provided with a third coating layer of 25% podophyllotoxin and 75% poly n-butymethacrylate-co-vinylacetate. As shown in FIG. 7, the result is a more rapid initial elution rate and followed by a slower, steady elution rate.

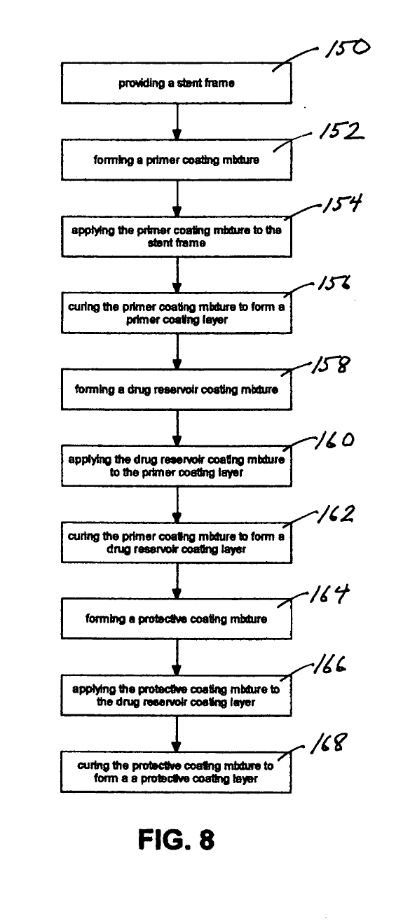

FIG. 8 shows a flow chart of a method of manufacturing a stent having an intermittent coating made in accordance with the present invention. A stent frame is provided at 150. A primer coating mixture is formed 152, the primer coating mixture applied to the stent frame 154, and the primer coating mixture cured to form a primer coating layer 156. A drug reservoir coating mixture is formed 158, the drug reservoir coating mixture applied to the primer coating layer 160, and the drug reservoir coating mixture cured to form a drug reservoir coating layer 162. A protective coating mixture is formed 164, the protective coating mixture applied to the drug reservoir coating layer 166, and the protective coating mixture cured to form a protective coating layer 168. In one embodiment, the stents can be checked with a microscope and weighed to assure the stents meet specifications. For commercial production, any stents with webbing, pooling, or weight outside the specification can be rejected.

The following provides specific examples of a cleaning procedure and the process of preparing a first coating layer 132, a second coating layer 134, and a third coating layer 136 for a stent having a sandwich type coating according to the present invention.

### EXAMPLE 1

To clean the stent frames prior to coating, stent frames made of 316LS stainless steel were placed in a carousel loading device to hold the stent frames secure and allow liquid contact with the stent frames. The loading device was then placed in a glass beaker. The beaker was filled with hexane to completely cover the stent frames and agitated in an ultrasonic bath for 15 minutes. After removing the beaker from the bath and discarding the hexane, the beaker was filled with 2-propanol to completely cover the stent frames and agitated in an ultrasonic bath for 15 minutes. After removing the beaker from the bath and discarding the 2-propanol, the beaker was filled with sodium hydroxide solution (1.0 N) to completely cover the stent frames and agitated in an ultrasonic bath for 15 minutes. After removing the beaker from the bath and discarding the sodium hydroxide solution, the stent frames were thoroughly rinsed with distilled water and dried in a vacuum oven overnight at 40° C.

### EXAMPLE 2

To produce an amino-silane first coating layer (primer coating) using an organic solvent, 0.2 g trimethoxysilyl-propyidiethylene-triamine (United Chemical Technology) amino-silane was weighed into a small vial, 17.6 ml CH3CN (acetonitrile) added to the same vial, and 6.7 ml tetrahydrofuran (THF) added to the same vial. After mixing the solution over a roller mixer for 15 minutes, the mixed solution was transferred into an auto-sonic spray machine. The auto-sonic spray machine sprayed a coating on the stent frames, which had been cleaned according to the method of Example 1, according to a pre-set program. The pre-set program controls the amount of coating dispensed, actual coating weight, coating uniformity, and coating process environment, such as humidity and temperature. The coated stents were dried in a hood for 30 minutes and then dried in a vacuum oven overnight at 40° C.

### EXAMPLE 3

To produce a vinylsilane first coating layer (primer coating) by a dipping method, a cleaned stent was placed in the 5% vinylsaline tetrahydrofuran (THF)/acetonitrile solution under ultra-sonic bath for 3 minutes. The salinized stent was washed with deionized water several times, and then placed at vacuum oven at 40° C. over night.

### EXAMPLE 4

To produce an amino-silane first coating layer (primer coating) with a polymer reservoir containing a drug or therapeutic agent, stent frames were first pre-weighed using a microbalance. The stent frames had been cleaned according to the method of Example 1. The following were mixed in a small vial: 0.1003 grams of trimethoxysilyi-propyldiethylene-triamine (United Chemical Technology) amino-silane, 0.1004 g rams of Resten-NG (AVI-4126) antisense compound, 4.75 ml of methanol, 10.1 ml of chloroform, and 1 ml of de-ionized water. After mixing the solution over a roller mixer, the mixed solution was transferred into an auto-sonic spray machine. The auto-sonic spray machine sprayed a coating on the stent frames according to a pre-set program. The pre-set program controls the amount of coating dispensed, actual coating weight, coating uniformity, and coating process environment, such as humidity and temperature. The coated stents were dried in a vacuum oven overnight at 40° C. The coated stents were weighed using a microbalance and the post-weight compared to the pre-weight to determine the weight of the first coating layer applied. The stents were checked under microscope and the weight compared with specifications.

### EXAMPLE 5

To produce a biodegradable polymer second coating layer (main drug reservoir coating), the stents were first pre-weighed using a microbalance. The stents had a first coating layer applied. A 100 ml volumetric flask was filled with tetrahydrofuran (THF). Five drug bottles of the drug etoposide containing about 100 mg etoposide per bottle were labeled, weighed, and the individual pre-weight of each drug bottle recorded. Inside a hood, a few ml THF from the volumetric flask was added to the first drug bottle, rinsing the inside of the neck of the bottle with the THF. The first drug bottle was then shaken to dissolve the etoposide. A pipette was used to transfer the etoposide/THF solution from the first drug bottle into a 200 ml small neck glass bottle, the small neck glass bottle having previously been cleaned with soapy water followed by THF. The first drug bottle was rinsed with THF twice and the etoposide/THF solution transferred by pipette twice to assure the all the etoposide was transferred to the small neck glass bottle. The procedure was repeated for the second through fifth drug bottles with their etoposide and the THF rinse transferred to the small neck glass bottle. Any THF remaining in the volumetric flask was also added to the small neck glass bottle. The five drug bottles were left open in the hood to allow any THF to evaporate and then re-capped. The five drug bottles were removed from the hood, weighed to determine their post-weight.

The total amount of etoposide transferred was calculated to be 0.4895 g by taking the difference between the pre- and post-weight. An equivalent weight of 0.4890 g of the bioabsorbable polymer polycaprolactone (PCL) was weighed out and added to the small neck glass bottle. The total volume of THF required to dissolve the drug and PCL to 1% of total solid concentration was calculated as 109 ml. An additional 9 ml THF was added to the 100 ml already present in the small neck glass bottle to reach the total 109 ml THF. The etoposide, PCL, and THF solution in the small neck glass bottle was shaken until all the drug and PCL polymer dissolved. The mixed solution was transferred into an auto-sonic spray machine, which sprayed a coating on the stents according to a pre-set program. The pre-set program controls the amount of coating dispensed, actual coating weight, coating uniformity, and coating process environment, such as humidity and temperature. The coated stents dried in a nitrogen atmosphere in an isolator overnight. The coated stents were weighed using a microbalance and the post-weight compared to the pre-weight to determine the weight of the second coating layer applied. The stents were checked under microscope and the weight compared with specifications.

### EXAMPLE 6

To produce a biodegradable polymer third coating layer (protective coating), the stents were first pre-weighed using a microbalance. The stents had first and second coating layers applied. A volume of 13 ml of chloroform was added to 0.2243 g DL-polylactide biodegradable polymer in a small glass vial. The solution was shaken until the DL-polylactide polymer dissolved. The mixed solution was transferred into an auto-sonic spray machine, which sprayed a coating on the stents according to a pre-set program. The pre-set program controls the amount of coating dispensed, actual coating weight, coating uniformity, and coating process environment, such as humidity and temperature. The coated stents dried in a nitrogen atmosphere in an isolator overnight. The coated stents were weighed using a microbalance and the post-weight compared to the pre-weight to determine the weight of the third coating layer applied. The stents were checked under microscope and the weight compared with specifications.

### EXAMPLE 7

To produce a non-biodegradable polymer second coating layer (main drug reservoir coating), the stents were first pre-weighed using a microbalance. The stents had a first coating layer applied. A weight of 0.0761 g of podophyllotoxin drug was weighed into a small glass vial. A weight of 0.0776 g of polyurethane Pellethane 80A (Dow Chemical Company) was weighed in a weighing boat, and then added to the small glass vial containing the podophyllotoxin. A volume of 16.1 ml of chloroform was added to the small glass vial and the small glass vial shaken until podophyllotoxin drug and Pellethane 80A polymer dissolved. In a second vial, 7.6 ml of methanol was added to 0.1490 g of poly (hydroxy ethylmethacrylate) polymer (PHEMA) and the second vial shaken until PHEMA polymer dissolved. The podophyllotoxin/Pellethane 80A solution and the PHEMA solution were combined and shaken well. The mixed solution was transferred into an auto-sonic spray machine, which sprayed a coating on the stents according to a pre-set program. The pre-set program controls the amount of coating dispensed, actual coating weight, coating uniformity, and coating process environment, such as humidity and temperature. The coated stents dried in a nitrogen atmosphere in an isolator overnight. The coated stents were weighed using a microbalance and the post-weight compared to the pre-weight to determine the weight of the second coating layer applied. The stents were checked under microscope and the weight compared with specifications.

### EXAMPLE 8

To produce a non-biodegradable polymer third coating layer (protective coating), the stents were first pre-weighed using a microbalance. The stents had first and second coating layers applied. A volume of 5 ml methanol was added to 0.1309 g of poly (hydroxy ethylmethacrylate) (PHEMA) polymer in a small vial. The PHMA solution was shaken until the PHMA polymer dissolved. In another vial, 10.6 ml chloroform was added to 0.0726 g of polyurethane Pellethane 80A (Dow Chemical Company). The Pellethane 80A solution was shaken until the Pellethane 80A polymer dissolved. The Pellethane 80A solution and the PHMA solution were combined and shaken well. The mixed solution was transferred into an auto-sonic spray machine, which sprayed a coating on the stents according to a pre-set program. The pre-set program controls the amount of coating dispensed, actual coating weight, coating uniformity, and coating process environment, such as humidity and temperature. The coated stents dried in a nitrogen atmosphere in an isolator overnight. The coated stents were weighed using a microbalance and the post-weight compared to the pre-weight to determine the weight of the third coating layer applied. The stents were checked under microscope and the weight compared with specifications.

### EXAMPLE 9

To produce a second and third coating layer, a first layer coating was applied to the stent using the method discussed in Example 2. A weight of 0.2109 g of podophyllotoxin was weighed into a g lass bottle. A weight of 0.6345 g of poly n-butymethacrylate-co-vinylacetate, 60:40, was weighed in a weighing pan and transferred into the bottle. A volume of 89 ml of tetrahydrofuran (THF) was added to the bottle and the bottle shaken until all drug and polymer dissolved. The solution was applied to the stent to form a second coating layer. To form a third coating layer, 0.2005 g of poly n-butymethacrylate-co-vinylacetate, 60:40, was placed in a glass bottle and 27.6 ml acetone added. The solution was shaken until polymer dissolved and applied to the stent to form a third coating layer. The solution was applied to the stent to form a third coating layer.

### EXAMPLE 10

To produce a second and third coating layer, a first layer coating was applied to the stent using the method discussed in Example 2. A weight of 0.2952 g of podophyllotoxin was weighed into a small glass vial. A weight of 0.2954 g of poly n-butymethacrylate-co-vinylacetate, 60:40, was weighed in a weighing pan and transferred into the glass vial. A volume of 65.8 ml of tetrahydrofuran (THF) was added to the bottle and the bottle shaken until all drug and polymer dissolved. The solution was applied to the stent to form a second coating layer. To form a third coating layer, 0.02243 g of poly n-butymethacrylate-co-vinylacetate, 60:40, was placed in a small glass vial. A weight of 0.06722 g of poly n-butymethacrylate-co-vinylacetate, 60:40, was weighed in a weighing pan and transferred into the glass vial. A volume of 11.2 ml of acetone was added. The solution was shaken until polymer dissolved and applied to the stent to form a third coating layer.

It is important to note that FIGS. 1-8 and the examples presented herein illustrate specific applications and embodiments of the present invention, and are not intended to limit the scope of the present disclosure or claims to that which is presented therein. For example, many combinations of materials and therapeutic agents can be used in the first, second, and third coating layers to achieve desired stent frame adherence, drug delivery timing, drug release profile and coating protection. In addition, many manufacturing methods using the combinations of solvents, polymers, and therapeutic agents can be used to manufacture the first, second, and third coating layers. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A stent delivery system comprising:
a catheter;
a balloon operably attached to the catheter; and
a stent disposed on the balloon;
wherein the stent comprises a stent frame, a primer coating layer disposed on the stent frame, a drug reservoir coating layer disposed on the primer coating layer, and a protective coating layer disposed on the drug reservoir coating layer.

2. The stent delivery system of claim 1 wherein the primer coating layer includes a therapeutic agent.

3. The stent delivery system of claim 1 wherein the protective coating layer includes a therapeutic agent.

4. A stent comprising:
a stent frame;
a primer coating layer disposed on the stent frame;
a drug reservoir coating layer disposed on the primer coating layer; and
a protective coating layer disposed on the drug reservoir coating layer.

5. The stent of claim 4 wherein material for the primer coating layer is selected from the group consisting of silanes, vinyltris(methylethylketoxime)silane, 2-(diphenylphosphino)ethyltriethoxysilane, 3-(1-aminopropoxy)-3,3-dimethyl-1-propenyl-trimethoxysilane, (aminoethylaminomethyl)phenethyltrimethoxysilane, 3-methacryloxypropyltrimethoxysilane, trimethoxysilyl-propyldiethylene-triamine, trichlorovinylsilane, 3-isocyanyopropyltriethoxysilane, 5-hexenyltrimethoxysilane, silicone polymers, acrylate polymers, epoxy type polymers, carboxylic polymers, polysulfide, phenolic resin, amino resin, polyurethane, polyvinyl acetate, polyvinyl alcohol, polyvinyl acetal, cyanoacrylate, polyester, polyamide, and combinations thereof.

6. The stent of claim 4 wherein the primer coating layer includes a therapeutic agent.

7. The stent of claim 6 wherein the therapeutic agent is selected from the group consisting of antiangiogenesis agents, antiendothelin agents, antimitogenic factors, antioxidants, antiplatelet agents, antiproliferative agents, antisense oligonucleotides, antithrombogenic agents, antibiotics, anti-inflammatory agents, antiinfective agents, antidiabetic agents, antiarteriosclerotics, antiarythmics, calcium channel blockers, clot dissolving enzymes, growth factors, growth factor inhibitors, nitrates, nitric oxide releasing agents, vasodilators, virus-mediated gene transfer agents, agents having a desirable therapeutic application, abciximab, angiopeptin, colchicine, eptifibatide, heparin, hirudin, lovastatin, methotrexate, Resten-NG (AVI-4126) antisense compound, streptokinase, ticlopidine, tranilast, sulindac, etoposide, podophyllotoxin, 5-fluorouracil, tissue plasminogen activator, trapidil, urokinase, growth factors, VEGF, TGF-beta, IGF, PDGF, FGF, and combinations thereof.

8. The stent of claim 4 wherein the drug reservoir coating layer is a biodegradable polymer.

9. The stent of claim 8 wherein the biodegradable polymer is selected from the group consisting of polycaprolactone, polylactide, polyglycolide, polyorthoesters, polyanhydrides, poly(amides), poly(alkyl 2-cyanocrylates), poly(dihydropyrans), poly(acetals), poly(phosphazenes), poly(dioxinones), trimethylene carbonate, polyhydroxybutyrate, polyhydroxyvalerate, similar polymers, their blends and copolymers and copolymers blends, and combinations thereof.

10. The stent of claim 4 wherein the drug reservoir coating layer is a non-biodegradable polymer.

11. The stent of claim 10 wherein the non-biodegradable polymer is selected from the group consisting of non-biodegradable hydrophobic polymers, non-biodegradable hydrophilic polymers, polyolefins, polystyrene, polyester, polysulfide, polyurethanes, polyacrylates, silicone polymers, cellulose polymers, polyvinyl polymers, polyvinyl alcohol and derivatives, polyvinyl pyrrolidone, polyethylene oxide, poly(hydroxy, aklymethacrylate), similar polymers, blends and copolymers and copolymers blends thereof, and combinations thereof.

12. The stent of claim 4 wherein the drug reservoir coating layer includes a therapeutic agent selected from the group consisting of antiangiogenesis agents, antiendothelin agents, antimitogenic factors, antioxidants, antiplatelet agents, antiproliferative agents, antisense oligonucleotides, antithrombogenic agents, antibiotics, anti-inflammatory agents, antiinfective agents, antidiabetic agents, antiarteriosclerotics, antiarythmics, calcium channel blockers, clot dissolving enzymes, growth factors, growth factor inhibitors, nitrates, nitric oxide releasing agents, vasodilators, virus-mediated gene transfer agents, agents having a desirable therapeutic application, abciximab, angiopeptin, colchicine, eptifibatide, heparin, hirudin, lovastatin, methotrexate, Resten-NG (AVI-4126) antisense compound, streptokinase, ticlopidine, tranilast, sulindac, etoposide, podophyllotoxin, 5-fluorouracil, tissue plasminogen activator, trapidil, urokinase, growth factors, VEGF, TGF-beta, IGF, PDGF, FGF, and combinations thereof.

13. The stent of claim 4 wherein the protective coating layer is a biodegradable polymer.

14. The stent of claim 13 wherein the biodegradable polymer is selected from the group consisting of polycaprolactone, polylactide, polyglycolide, polyorthoesters, polyanhydrides, poly(amides), poly(alkyl 2-cyanocrylates), poly(dihydropyrans), poly(acetals), poly(phosphazenes), poly(dioxinones), trimethylene carbonate, polyhydroxybutyrate, polyhydroxyvalerate, similar polymers, blends and copolymers and copolymers blends thereof, and combinations thereof.

15. The stent of claim 4 wherein the protective coating layer is a non-biodegradable polymer.

16. The stent of claim 15 wherein the non-biodegradable polymer is selected from the group consisting of non-biodegradable hydrophobic polymers, non-biodegradable hydrophilic polymers, polyolefins, polystyrene, polyester, polysulfide, polyurethanes, polyacrylates, silicone polymers, cellulose polymers, polyvinyl polymers, polyvinyl alcohol and derivatives, polyvinyl pyrrolidone, polyethylene oxide, poly(hydroxy, aklymethacrylate), similar polymers, blends and copolymers and copolymers blends thereof, and combinations thereof.

17. The stent of claim 4 wherein the protective coating layer includes a therapeutic agent.

18. The stent of claim 17 wherein the therapeutic agent is selected from the group consisting of antiangiogenesis agents, antiendothelin agents, antimitogenic factors, antioxidants, antiplatelet agents, antiproliferative agents, antisense oligonucleotides, antithrombogenic agents, antibiotics, anti-inflammatory agents, antiinfective agents, antidiabetic agents, antiarteriosclerotics, antiarythmics, calcium channel blockers, clot dissolving enzymes, growth factors, growth factor inhibitors, nitrates, nitric oxide releasing agents, vasodilators, virus-mediated gene transfer agents, agents having a desirable therapeutic application, abciximab, angiopeptin, colchicine, eptifibatide, heparin, hirudin, lovastatin, methotrexate, Resten-NG (AVI-4126) antisense compound, streptokinase, ticlopidine, tranilast, sulindac, etoposide, podophyllotoxin, 5-fluorouracil, tissue plasminogen activator, trapidil, urokinase, growth factors, VEGF, TGF-beta, IGF, PDGF, FGF, and combinations thereof.

19. The stent of claim 4 wherein the drug reservoir coating layer and the protective coating layer are made of the same polymer.

20. The stent of claim 4 wherein the drug reservoir coating layer and the protective coating layer are made of different polymers.

21. A method of manufacture of a stent comprising:
providing a stent frame;
forming a primer coating mixture;
applying the primer coating mixture to the stent frame;
curing the primer coating mixture to form a primer coating layer;
forming a drug reservoir coating mixture;
applying the drug reservoir coating mixture to the primer coating layer;
curing the primer coating mixture to form a drug reservoir coating layer;
forming a protective coating mixture;
applying the protective coating mixture to the drug reservoir coating layer; and
curing the protective coating mixture to form a protective coating layer.

22. The method of claim 21 wherein forming a primer coating mixture comprises mixing a silane and a solvent.

23. The method of claim 21 wherein forming a primer coating mixture comprises mixing a silane, a therapeutic agent, and a solvent.

24. The method of claim 21 wherein forming a primer coating mixture comprises mixing a polymer and a solvent.

25. The method of claim 21 wherein forming a primer coating mixture comprises mixing a polymer, a therapeutic agent, and a solvent.

26. The method of claim 21 wherein applying the primer coating mixture to the stent frame further comprises applying the primer coating layer by a method selected from the group consisting of spraying, dipping, brushing, painting, wiping, vapor deposition, plasma deposition, electrostatic deposition, epitaxial growth, and combinations thereof.

27. The method of claim 21 forming a drug reservoir coating mixture comprises mixing a polymer, a therapeutic agent, and a solvent.

28. The method of claim 21 wherein applying the drug reservoir coating mixture to the primer coating layer further comprises applying the drug reservoir coating layer by a method selected from the group consisting of spraying, dipping, brushing, painting, wiping, vapor deposition, plasma deposition, electrostatic deposition, epitaxial growth, and combinations thereof.

29. The method of claim 21 wherein forming a protective coating mixture comprises mixing a polymer and a solvent.

30. The method of claim 21 wherein forming a primer coating mixture comprises mixing a polymer, a therapeutic agent, and a solvent.

31. The method of claim 21 wherein applying the protective coating mixture to the drug reservoir coating layer further comprises applying the protective coating layer by a method selected from the group consisting of spraying, dipping, brushing, painting, wiping, vapor deposition, plasma deposition, electrostatic deposition, epitaxial growth, and combinations thereof.

32. A system for producing a stent comprising:
means for forming a primer coating mixture;
means for applying the primer coating mixture to a stent frame;
means for curing the primer coating mixture to form a primer coating layer;
means for forming a drug reservoir coating mixture;
means for applying the drug reservoir coating mixture to the primer coating layer;
means for curing the primer coating mixture to form a drug reservoir coating layer;
means for forming a protective coating mixture;
means for applying the protective coating mixture to the drug reservoir coating layer; and
means for curing the protective coating mixture to form a protective coating layer.

33. A stent comprising:
a stainless steel stent frame;
a first coating layer disposed on the stainless steel stent frame, material of the first coating layer being selected from the group consisting of silanes, vinyltris(methylethylketoxime)silane, 2-(diphenylphosphino)ethyltriethoxysilane, 3-(1-aminopropoxy)-3,3-dimethyl-1-propenyl-trimethoxysilane, (aminoethylaminomethyl)phenethyltrimethoxysilane, 3-methacryloxypropyltrimethoxysilane, trimethoxysilyl-propyldiethylene-triamine, trichlorovinylsilane, 3-isocyanyopropyltriethoxysilane, 5-hexenyltrimethoxysilane, silicone polymers, acrylate polymers, epoxy type polymers, carboxylic polymers, polysulfide, phenolic resin, amino resin, polyurethane, polyvinyl acetate, polyvinyl alcohol, polyvinyl acetal, cyanoacrylate, polyester, polyamide, and combinations thereof;
a second coating layer disposed on the first coating layer, the second coating layer comprising a therapeutic agent and a material selected from the group consisting of polycaprolactone, polylactide, polyglycolide, polyorthoesters, polyanhydrides, poly(amides), poly(alkyl 2-cyanocrylates), poly(dihydropyrans), poly(acetals), poly(phosphazenes), poly(dioxinones), trimethylene carbonate, polyhydroxybutyrate, polyhydroxyvalerate, non-biodegradable hydrophobic polymers, non-biodegradable hydrophilic polymers, polyolefins, polystyrene, polyester, polysulfide, polyurethanes, polyacrylates, silicone polymers, cellulose polymers, polyvinyl polymers, polyvinyl alcohol and derivatives, polyvinyl pyrrolidone, polyethylene oxide, poly(hydroxy, aklymethacrylate), similar polymers, blends and copolymers and copolymers blends thereof, and combinations thereof; and
a third coating layer disposed on the second coating layer, material of the third coating layer being selected from the group consisting of polycaprolactone, polylactide, polyglycolide, polyorthoesters, polyanhydrides, poly(amides), poly(alkyl 2-cyanocrylates), poly(dihydropyrans), poly(acetals), poly(phosphazenes), poly(dioxinones), trimethylene carbonate, polyhydroxybutyrate, polyhydroxyvalerate, non-biodegradable hydrophobic polymers, non-biodegradable hydrophilic polymers, polyolefins, polystyrene, polyester, polysulfide, polyurethanes, polyacrylates, silicone polymers, cellulose polymers, polyvinyl polymers, polyvinyl alcohol and derivatives, polyvinyl pyrrolidone, polyethylene oxide, poly(hydroxy, aklymethacrylate), similar polymers, blends and copolymers and copolymers blends thereof, and combinations thereof.
